(19) United States
(12) Patent Application Publication
(10) Pub. No.: US 2003/0211139 A1
(43) Pub. Date: Nov. 13, 2003

(54) Dispersions of lipid particles for use as therapeutic and cosmetic agents and intracellular delivery vehicles

(76) Inventors: Thierry Legon, Korbeek-Lo (BE); Frederic Henot, Brussels (BE)

Correspondence Address: MERCHANT & GOULD PC, P.O. BOX 2903, MINNEAPOLIS, MN, 55402-0903, US

(21) Appl. No.: 10/140,369

(22) Filed: May 7, 2002

(51) Int. Cl. A61K 48/00; A61K 9/127

(52) U.S. Cl. 424/450; 514/2; 514/44

(57) ABSTRACT

In a dispersion of lipid particles in a dispersing medium, the said lipid particles comprising an amino-amidine compound A, the amidine function of the said compound A is titrated substantially in water by means of an acid HX, wherein X is an anion, in a manner such that the pH of the said lipid dispersion is between about 6.5 and 7.8 within a temperature range from about 2° C. to 40° C. The so titrated dispersion is useful inter alia as a component of a synthetic vector for therapeutic molecules or macromolecules.

The present invention relates to non-viral delivery systems for therapeutic agents. More specifically this invention relates to lipid particles dispersions, in particular liposomes, having long-term stability and having a pH compatible with that of physiological solutions. The present invention further relates to a method for making such dispersions and for making solid compositions therefrom. These dispersions are useful as components of synthetic vectors for therapeutic molecules or macromolecules such as DNA, proteins and polypeptides and therefore useful for introducing such molecules into eukaryotic cells. The invention also relates to cells transformed by means of such synthetic vectors as well as to pharmaceutical compositions comprising effective amounts thereof.

### BACKGROUND OF THE INVENTION

Liposomes may be defined as vesicles in which an aqueous volume is entirely enclosed by a bilayer membrane composed of lipid molecules. When dispersing these lipids in aqueous media, a population of liposomes with sizes ranging from about 15 nm to about 1 μm may be formed. The three major types of lipids, i.e. phospholipids, cholesterol and glycolipids, are amphipathic molecules which, when surrounded on all sides by an aqueous environment, tend to arrange in such a way that the hydrophobic “tail” regions orient toward the center of the vesicle while the hydrophilic “head” regions are exposed to the aqueous phase. According to this mechanism liposomes thus usually form bilayers.

Several types of liposomes are known in the art. Referring to their physical structure, the more simple type of liposomes to prepare consists of multilamellar vesicles (hereinafter referred to as MLV, according to standard practice in the art), i.e. onion-like structures characterized by multiple membrane bilayers, each separated from the next by an aqueous layer, usually having a size between about 100 nm and 1 ∥m. Their production can be reproducibly scaled-up to large volumes and they are mechanically stable upon storage for long periods of time. Contrary to this, small unilamellar vesicles (hereinafter referred to as SUV, according to standard practice in the art) usually having a size between about 15 nm and 200 nm, possess a single bilayer membrane and are usually difficult to prepare on a large scale because of the high energy input required for their production and of the risks of oxidation and hydrolysis. In addition, SUV are thermodynamically unstable and are susceptible to aggregation and fusion. Furthermore, as the curvature of the membrane increases in SWN. It develops a degree of asymmetry, i.e. the restriction in packing geometry dictates that significantly more than 50% and up to 70% of the lipids making up the bilayer are located on the outside. Because of this asymmetry, the behaviour of SUV is markedly different from that of bilayer membranes comprising MLV or from that of large unilamellar vesicles (the latter, hereinafter referred to as LUV, usually having a size between about 100 nm and 1 μm).

Referring to their chemical structure, liposomes may be made from neutral phospholipids, negatively-charged (acidic) phospholipids, sterols and other non-structural lipophillc compounds. For instance, EP-B-165,680 discloses steroidal liposomes comprising completely closed bilayers substantially comprising a salt form of an organic acid derivative of a sterol. A population of detergent-free liposomes having a substantially unimodal distribution (i.e. unilamellar vesicles) about a mean diameter greater than 50 nm and exhibiting less than a twofold variation in size may be produced, according to EP-B-185,756, by first preparing multilamellar liposomes and then repeatedly passing the liposomes under pressure through a filter having a pore size not more than 100 nm. Multilamellar vesicles are known from U.S. Pat. No. 4,522,803 and U.S. Pat. No. 4,558,579. A process for improving the trapping efficiency of multilamellar vesicles, comprising repeated freezing at −196° C. and warming in a constant temperature bath, is also disclosed by EP-B-231,201. For a detailed description of liposomes and methods of manufacturing them, reference is hereby made to Liposomes, a practical approach (1990), Oxford University Press. WO 89103679 discloses the production of liposomes comprising the salt form of a pH sensitive lipid being an organic acid derivative of a sterol or a tocopherol. WO 95/17378 discloses positively charged vesicles for combination with nucleic acids, polypeptides or proteins, comprising a compound having an amidine group. More specifically, this document shows efficiencies of 60 to 68% when transfecting Chinese hamster Ovary cells or K562 human myeloid cells by means of MLV consisting of 3-tetradecylamino-N-terbutyl-N′-tetradecylpropionamidine. However, it appears that certain other cell lines, for instance fibroblasts such as COS-7 monkey fibroblasts or NIH-3T3 mouse fibroblasts, are not appropriately transfected by means of MLV comprising the amino-amidine compounds of the prior art. This indicates that, within the family of amino-amidine compounds used for preparing liposomes for intacellular delivery of genetic material into eukaryotic cells, a need exists in the art for modifications of the said compounds and/or the physical structure of vesicles including them in order to allow for appropriate transfection of a wide range of cells, including certain types of cells such as the above-mentioned fibroblasts, and optionally to render the said vesicles sensitive to the pH shift occurring during their introduction into the cell. A need also exists in the art for producing now types of vesicles comprising such compounds which would be mechanically stable upon storage for long periods of time while retaining their pH-sensitivity characteristics.

Attempts have been made to produce vesicles or liposomes from the amino-amidine compounds of the prior art in the presence of an organic hydrophobic polyfunctional buffer for instance taken from the classes of aminosulfonic acids or hydroxylated amines. For instance Defrise-Quertain et al. in J. Chem. Soc. Chem Commun. (1986) 1060-1062 discloses producing dispersions, similar to liposome suspensions and having a bilayer organization, by vortex mixing 3-tetradecyclamino-N-terbutyl-N′-tetradecylpropionamidine (having a melting point of 34° C.) In hot water (40-70° C.) optionally in the presence of a tris(hydroxymethyl)aminoethane/HCl buffer and, upon sonication, decreasing the hydrodynamic diameter of the vesicles down to 87 nm. Unfortunately, repeating this manufacturing procedure has evidenced the problems of (I) yielding dispersions of which the pH is not compatible with physiological pH within the temperature range useful for most medical applications, and (II) yielding dispersions which readily precipitate after a few hours storage at low temperature. Pector et al. in Biochimica et Biophysicia Acta (1998).339-346 discloses forming liposomes of 3-tetradecylamino-N-terbutyl-N′-tetradecylpropionamidine after addition of a buffer comprising N-2-hydroxyethylpiperazine-N′-2-ethanesulfonic acid at pH 7.3 and mechanical mixing above 23° C., then titrating the said liposomes at various saline concentrations by means of 0.1 M HCl. Due to the fact however that the organic buffer anion being present in the latter procedure is able to interact with any positively charged amidino group, the disclosed procedure necessarily achieves a poorly defined mixture of lipidic particles that is not suitable for further handling, storage and use as intracellular delivery vehicles, therefore teaching away from the manufacture of vectors introducing molecules and macromolecules into a cell. Therefore there is a need in the art for well-defined dispersions, e.g. cationic liposomes, based on compounds having an amidine function that would be suitable as intracellular delivery vehicles. In particular, there is a need in the art for such aqueous dispersions which would have a pH compatible with physiological pH within the temperature range useful for most medical applications, e.g. between about 2° C. and 40° C. There is also a need in the art for dispersions which combine such an amino-amidine compound with another type of lipid while keeping the above-mentioned advantageous characteristics. For economical reasons, such as decreasing the volume and weight of the useful biological material and hence its cost of transportation, there is also a need in the art for solid compositions such as lyophilisates or amorphous particles, which are able to achieve and retain the above-mentioned advantageous characteristics when dispersed in a liquid medium such as water or an appropriate buffer.

### SUMMARY OF THE INVENTION

The present invention is based on several unexpected findings. First, well-defined dispersions of lipid particles, such as liposomes, based on compounds having an emidine function can be obtained, which are capable of retaining a pH compatible with physiological pH within a temperature range useful for most medical applications, e.g. between about 20° C. and 40° C. Another aspect of the invention is the ability of the said liposomes based on compounds having an amidine function to efficiently transfect in vitro or in vivo a wide range of types of cells. Secondly and quite importantly, the pH characteristics of the lipid particles dispersions of the invention are not adversely affected when the said dispersions are dried or freeze-dried into a solid composition and the said solid composition is thereafter redispersed in another aqueous medium such as for instance an organic functional buffer. Consequently, the dispersions of the invention are useful for making synthetic vectors for combining with a wide range of biologically active molecules, especially far complexing or entrapping macromolecular and/or biodegradable substrates. Further, the resulting synthetic vectors are effective for introducing the said biologically active molecule or macromolecule into a wide range of eukaryotic cells. This invention further includes methods for introducing biologically active molecules into eukaryotic cells, as well as eukaryotic cells transformed by means of the aforesaid synthetic vectors and pharmaceutical compositions comprising effective amounts of the synthetic vectors. The said pharmaceutical compositions are useful for the prophylactic or therapeutic treatment of mammals for a wide range of diseases and disorders, depending on the biological activity of the relevant molecule or macromolecule. The invention also includes various uses of the said lipid particle dispersions, such as for instance as an anti-microbial agent, an anti-inflammatory agent, a cosmetic agent, an emulsifier, a detergent, a vaccine adjuvant or a diagnostic reagent.

### BRIEF DESCRIPTIION OF THE DRAWINGS

FIG. 1 shoves the variation of pH at 25° C. as a function of the amount of hydrochloric acid added during titration of amino-amidine liposomes in water (Injection grade available from Baxter, cat. Nr. ADA 0304).

FIG. 2 shows the variation of pH at 25° C. as a function of the amount of hydrochloric acid added during titration of amino-amidine liposomes in sodium phosphate buffer.

Definitions

“Transfection” is defined herein as the intracellular delivery of active biological material, especially genetic material (such as defined hereinafter) into the cells of a eukaryotic organism, preferably a mammal, and more preferably, a human. The said genetic material is preferably expressible and produces beneficial proteins after being introduced into the cell. Alternatively, the said genetic material is used to bind to or interact with a site within the cell, or encodes a material that binds to or interacts with a site within the cell. When a virus binds to and enters cells via polyanionic sites on the cell surface. Increasing or decreasing transfection efficiency also affects viral infection. Suitable cell types that can be transfected using this invention include, but are not limited to, cells performing endocytosis or phagocytosis, fibroblasts, myoblasts, hepatocytes, cells of hematopoetic origin such as white blood cells and bone marrow cells, cancer cells and ischemic tissue. Transfection can be performed in vitro, ex vivo, or in vivo. The genetic material can be transiently expressed or stably expressed.

In vitro transfection involves transfecting calls outside of a living eukaryotic organism, e.g. using cell cultures. In vivo transfection involves transfecting cells within a living eukaryotic organism. Ex vivo transfection involves removing cells from an organism, transfecting at least part of the cells, and returning the cells to the said organism.

The term “removing” as used herein refers to any method known to obtain a sample of living cells from a eukaryotic organism, including venipucture, call scraping, punch biopsy, needle biopsy and surgical excision. The term “returning” includes methods known to replace cells in the body of a mammal, preferably a human, such as intravenous introduction, surgical implantation and injection.

“Transient gene expression” is defined herein as temporary gene expression that diminishes over time under selective conditions, i.e. usually occurring over periods of less than one year to periods as short as one week. The gene therapy application, the vector construct, whether or not chromosome integration has occurred, the cell type and the location of cell implantation following transfection are known to influence the length of time that a particular gene is expressed.

“Stable gene expression” is defined herein as gene expression that does not significantly diminish over time, i.e. the transfected calls produce a relatively constant level of gene product for relatively long periods of time.

“Genetic material” is defined herein as DNA, RNA, mRNA, rRNA, tRNA, uRNA, ribozymes, antisense oligonucleotides, peptide nucleic acid (PNA), plasmid DNA or a combination thereof. Modified nucleosides can be incorporated into the genetic material in order to impart in vivo and in vitro stability of the oligonucleotides to endo- and exonucleases, alter the charge, hydrophilicity or lipophilicity of the molecule, and/or provide differences in three-dimensional structure.

As used herein, the term “C₁₋₈ alkyl” refers to straight and branched chain saturated hydrocarbon monovalent radicals or groups having from 1 to 8 carbon atoms such as, for example, methyl, ethyl, propyl, n-butyl, 1-methylethyl, 2-methylpropyl, 1,1-dimethylethyl, 2-methylbutyl, n-pentyl, dimethylpropyl, n-hexyl, 2-methylpentyl, 3-methylpentyl, n-heptyl, 2-ethylhexyl, n-octyl and the like.

As used herein, the term “C₃₋₁₀ cycloalkyl” refers to monocyclic or polycyclic aliphatic monovalent radicals or groups having from 3 to 8 carbon atoms, such as for instance cyclopropyl, 1-2-dimethylcyclopropyl, cyclobutyl, methylcyclobutyl, cyclopentyl, methylcyclopentyl, cyclohexyl, methylcyclohexyl, ethylcyclohexyl, cycloheptyl, cyclooctyl, norbomyl, adamantyl, and the like.

As used herein, the term “aryl” refers to mono- and polyaromatic monovalent radicals such as phenyl, naphtyl, anthracenyl, phenantracyl, fluoranthenyl, chrysenyl, pyrenyl, picenyl and the like, including fused benzo-C₅₋₈ cycloalkyl radicals such as, for instance, indanyl, 1,2,3,4-tetrahydronaphtalenyl, fluorenyl and the like,

As used herein, the term “heeroaryl” means a mono- and polyheteroaromatic monovalent radical including one or more heteroatoms selected from the group consisting of nitrogen, oxygen, sulfur and phosphorus, such as for instance pyridinyl, pyrazinyl, pyrimidinyl, pyridazinyl, triazinyl, triazolyl, imidazolyl, pyrazolyl, thiadazolyl, isothiazolyl, oxazolyl, pyrrolyl, furanyl, thienyl, indolyl, indazolyl, benzofuryl, benzothienyl, quinolyl, quinazolinyl, quinoxalinyl, carbazolyl, phenoxazinyl, phenothiazinyl, xanthenyl, purinyl and the like, including all possible isomeric forms thereof.

As used herein, the term “C₁₂₋₂₀ alkyl” refers to straight and branched chain saturated hydrocarbon monovalent radicals having from 12 to 20 carbon atoms such as, for example, dodecyl, tetradecyl, hexadecyl, octadecyl and the like.

As used herein, the term “C₁₋₂₀ alkyl” includes C₁₋₈ alkyl and C₁₂₋₂₀ alkyl (such as hereinabove defined) and homologues thereof having from 9 to 11 carbon atoms, such as for instance nonyl, decyl, undecyl and the like.

### DETAILED DESCRIPTION OF THE INVENTION

In a first embodiment, the present invention includes a dispersion of lipid particles comprising an amino-amidine compound A having the general formula:

R₁HN—(CH₂)ₙ—C(═NR₂)—NR₃R₄ (I)

wherein each of R₁, R₂, R₃ and R₄ is independently selected from the group consisting of hydrogen, C₁₋₂₀ alkyl, C₃₋₁₀ cycloalkyl, aryl and heteroaryl radicals, and n is a positive integer, and optionally one or more lipids B, the said dispersion being characterized in that the amidine function of the said compound A is titrated substantially in water by means of an acid HX, wherein X is an anion, in a manner such that the pH of the said lipid dispersion is between about 6.5 and 7.8 within a temperature range from about 2° C. to 40° C.

In view of the main uses of the said dispersion, such as detailed hereinafter, it is highly preferred that:

R₂ is a C₁₋₈ alkyl group, preferably a tert-butyl group,

n is an integer from 1 to 6 inclusive, preferably n=2, and

R₁ is a C₁₂₋₂₀ in alkyl group, one of R₃ and R₄ is hydrogen and the other of R₃ and R₄ is a C₁₂₋₂₀ alkyl group.

In a most preferred embodiment of this invention, the amino-amidine compound A is selected from the group consisting of N-terbutyl-N-tetradecyl-3-tetradecyl-aminopropionamidine, N-terbutyl-N′-dodecyl-3-dodecylaminopropionamidine, N-terbutyl-N′-hexadecyl-3-hexadecylaminopropionamidine and N-terbutyl-N′-octadecyl-3-octadecylaminopropionamidine. All amino-amidine compounds A failing under the above definition, especially those mentioned in the above most preferred embodiment, are either well known in the art or can be obtained by procedures and methods similar to the procedures used for preparing the well known compounds (i.e. by aminolysis of ethyl-N-terbutylacrylimidate with a fatty amine, see for instance D. G. Neilson ‘The Chemistry of Amidines and Imidates’ (1975), ed, S. Patai, Wiley, New-York, and R. Fuks, Bull. Soc. Chim. Belg. (1980) 89:433), while performing routine experimental work and changing the starting materials according to ordinary skill in the art.

The dispersion according to the first embodiment of the invention may include only, i.e. may consist of, an amino-amidine compound A having the general formula (I) or a mixture of such compounds, or alternatively it may further include one or more lipid(s) B. Such lipids may for instance be selected from the group consisting of phospholipids, sterols, tocopherols or other lipophilic compounds, preferably those which are already known in the art for their ability to form liposomes under appropriate conditions. Preferably the lipid B is a biocompatible lipid selected from the group consisting of fatty acids, lysolipids, phosphatidylcholines, phosphatidylethanolamines, phosphatidylserines, phosphatidylglycerols, phosphatidylinositols, sphingolipids (such as sphingomyelin), glycolipids (such as gangliosides), sulfatides, glycosphingolipids; lipids bearing functional moieties such as polyethyleneglycol, chitin, hyaluronic acid, polyvinylpyrrolidone, polylysine, polyarginine, sulfonated mono-, di- or oligosaccharides; cholesterols; sterol aliphatic acid esters (such as cholesterol butyrate, cholesterol isobutyrate, cholesterol palmitate, cholesterol stearate, lanosterol acetate, ergosterol palmitate, and phytosterol n-butyrate); dicetyl phosphates, stearylamines, cardiolipin, synthetic phospholipids with asymmetric acyl chains, ceramides, sterol aliphatic acid esters; sterol esters of sugar acids (such as cholesterol glucuronide, lanosterol glucuronide, 7-dehydrocholesterol glucuronide, ergosterol glucuronide, cholesterol gluconate, lanosterol gluconate, and ergosterol gluconate): esters of sugar acids and sugar alcohols (such as lauryl glucuronide, stearoyl glucuronide, myristoyl glucuronida, lauryl gluconate, myristoyl gluconate, and stearoyl gluconate); sugar esters and aliphatic add esters (such as sucrose laurate, fructose laurate, sucrose palmitate, sucrose stearate, glucuronic acid, gluconic acid, accharic acid, and polyuronic acid); saponins (such as sarsasapogenin, smilagenin, hederagenin, oleanolic acid, and digitoxigenin); glycerol esters (such as glycerol tripalmitate, glycerol distearate, glycerol tristearate, glycerol dimyristate, and glycerol trimyristate); alcohols having 10 to 30 carbon atoms (such as n-decyl alcohol, lauryl alcohol, myristyl alcohol, cetyl alcohol, and n-octadecyl alcohol), D-galactopyranosides, digalactosyldiglyceride, N-succinyldioleoylphosphatidylethanolamine, palmitoyl homocysteine, alkyl phosphonates, alkyl phosphinates and alkyl phosphites. Suitable phosphatidylcholines include dioleoylphosphatidylcholine, dimyristoylphosphatidylcholine, dipentadecanoylphosphatidylcholine, dilauroylphosphatidylcholine, dipalmitoylphosphatidylcholine and distearoylphosphatidylcholine.

The said lipid(s) B may be admixed with the amino-amidine compounds A in various proportions. The only restriction to the selection of the lipids B and of their proportion in the mixture is that they should not significantly affect or otherwise be detrimental to the advantageous properties of the lipid particles dispersions of the invention, i.e. namely providing well-defined liposomes which retain their pH characteristics (such as above defined) while being able to efficiently transfect a wide range of types of cells. Given the teachings of the present invention and the general knowledge in the art, the skilled person will be able in each case to determine whether a given lipid B and a given proportion for the latter meet these criteria. However, as a general rule, it should be understood that the amino-amidine compound A should preferably be the main organic component of the dispersion or the invention, i.e. the weight amount of the lipid(s) B should preferably be at most about 50%.

Titration of the amidine function of compound A substantially in water is an essential requirement of this first embodiment of the present invention. The term “substantially in water” as used herein means that, contrary to the teachings of the prior art, titration by means of an acid HX is not effected in the presence of one of these organic functional buffers, such as aminosulfonic hydroxylated amines, which were found to greatly interfere with liposomes stability. Although substantially pure water is a preferred embodiment of the present invention, a mineral buffer such as sodium or potassium phosphate or alkali metal salts of bicarbonate often does not significantly interfere with stability of the lipid particles dispersion obtained and may therefore be used in place of pure water. Another requirement of this first embodiment of the present invention is that titration of compound A should be performed until the pH of the lipid particles dispersion is between about 6.5 and 7.8 within a temperature range from about 2° C. to 40° C. Such further condition clearly contributes to obtaining a well chemically defined composition, as opposed to the poorly defined mixtures of salts and liposomes of the prior art. The skilled person knows how to meet this second condition, e.g. by accurately controlling the titration process, for instance by continuously measuring the pH of the dispersion during the addition of the add HX, by continuously processing the lipid particles and by interrupting the said addition as soon as the pH value within the required range is achieved and stable.

The anion X of the acid used for titration of the amidine function according to the first embodiment of the present invention may suitably be either that of a strong acid or a weak acid, these terms being understood according to their usual meaning in the chemical art as exemplified herein-below in a non exhaustive manner. A strong acid includes anions such as iodide, bromide, chloride, nitrate, perchlorate, sulfate, tosylate and methanesulfonate. A weak acid includes anions such as acetate, fluoride, borate, hypobromite, hypochlorite, nitrite hyponitrite, sulfite, phosphate, phosphate, phosphonate, chlorate, oxalate, malonate, succinate, lactate, carbonate, bicarbonate, benzoate, citrate, permanganate, manganate, propanoate, butanoate and chromate.

In view of the unexpected property of the lipid particles dispersions which constitute the first embodiment of the present invention, i.e. their advantageous pH characteristics are not altered by a change in the dispersing medium, they may take various forms. The lipid particles within the said dispersion are preferably liposomes. However they may also be amorphous solid particles or emulsion droplets. Preferably the dispersing medium of the lipid particles dispersion of the invention is an aqueous medium consisting of water being already present during titration of the amidine function of compound A. As previously mentioned, the said dispersing medium may also comprise a mineral buffer which may either be already present during titration of the amidine function or which may be added thereafter if need be for some specific applications. The said dispersing medium may also comprise an organic functional buffer. Suitable examples of such organic functional buffers are well known in the art of biology and include for instance aminosulfonic acids (such as N-2-hydroxyethylpiperazine-N′2-ethanesulfonic acid (HEPES), 3-(N-morpholino)propanesulfonic acid (MOPS), piperazine-N,N′-bis(2-ethanesulfonic acid (PIPES), N-[tris(hydroxymethyl)methyl]-3-amino-2-hydroxypropanesulfonic acid (TAPSO), piperazine-1,4-bis(2-hydroxy propanesulfonic acid) dihydrate (POPSO), N,N-bis(2-hydroxyethyl)-2-aminoethanesulfonic acid (BES). 2-[(2-hydroxy-1,1-bis[hydroxymethyl]ethyl)amino]ethanesulfonic acid (TES), N,N-bis(2-hydroxyethyl)-3-amino-2-hydroxypropanesulfonic acid (DIPSO), [(2-Hydroxy-1,1-bis[hydroxymethyl]ethyl)amino]-1-propanesulfonic acid (TAPS), 3-(cyclohexylamino)-1-propanesulfonic acid (CAPS), 4-(2-hydroxyethyl)piperazine-1-propanesulfonic acid (EPPS), 4-(2-hydroxyethyl)piperazine-1-(2-hydroxypropanesulfonic acid) monohydrate (HEPPSO), 2-(N-morpholino)ethanesulfonic acid and the like), hydrated amines (such as tris(hydroxymethyl)aminoethane, N,N-bis(2-hydroxyethyl)glycine (Bicine), N-(2-hydroxy-1,1-bis[hydroxymethyl]ethyl) glycine (Tricine), 1,3-bis[tris(hydroxymethyl) methylamino]propane and the like) and mixtures thereof.

The lipid particles according to the invention may also be in the form of an emulsion, i.e. for instance by combining a lipid particles aqueous dispersion with an oily component, the titrated compound A may act as a surfactant and contribute to the formation of a continuous lipid phase surrounded by an amino-amidine layer.

In a second embodiment, the present invention includes a method for making a dispersion of lipid particles such as defined hereinabove, comprising the steps of:

(a) dispersing an amino-amidine compound A having the general formula (I), optionally admixed with one or more lipid(s) B, in a liquid medium comprising an aqueous medium and optionally an organic solvent for compound A and for the lipid(s) B, and optionally an oily component,

(b) processing the dispersion obtained in step (a) until vesicles comprising compound A and optionally one or more lipid(s) B are obtained, and

(c) titrating the vesicles obtained in step (b) with an acid HX, wherein X is an anion, and

(d) optionally processing the titrated vesicles obtained in step (C) until pH stabilisation

the said method being characterized in that:

the aqueous medium of step (a) substantially consists of water, and

the acid HX is used in step (c) in an amount such as to substantially form an amidinium salt (A, HX) and such that the pH of the said lipid dispersion after titration is between about 6.5 and 7.8 within a temperature range from about 2° C. to 40° C.

A first important feature of the method of the invention is that, contrary to the teachings of the prior art, buffers which due to their chemical definition are likely to interfere with the desired chemical reaction involved during the titration step of the method should be avoided. Interference with titration should be understood to mean that the buffer induces particles aggregation or fusion. In practice, this means that the standard organic multifunctional buffers commonly used in the art of biology, such as the well known classes of aminosulfonic acids or hydroxylated amines previously mentioned, should be carefully avoided. Therefore the preferred aqueous medium to be used in step (b) of the method is water or, alternatively, a non-interfering buffer as previously disclosed.

A second important feature of the method of the invention is that the acid HX used for titration should be present in an amount sufficient but necessary to substantially and quantitatively form the desired amidinium salt (A. MX), i.e. substantially free of the free-amidine form of compound A. An improved working embodiment of the manufacturing method of the invention further includes, after step (c), the step of measuring and optionally adjusting the pH of the titrated liposomes until a pH between about 6.5 and 7.8 is obtained. This optional step may be used as a quality control step and is performed according to standard practice in the art.

Depending on the specific desired form of the lipid particle dispersion, variations of the manufacturing method of the invention may be as follows. First, liposomes will be obtained when the liquid medium used in step (a) is an aqueous medium, e.g. water or a non-interfering buffer. If desired, namely in the latter case, the method of the invention may further include the stop of admixing the lipid particles dispersion obtained after step (c) or step (d) with a buffer. The said buffer may be identical with or different from the mineral buffer (non-adversely interfering with liposomes stability) optionally present during titration. For instance it may be an organic functional buffer such as previously defined.

In order to obtain the dispersion in the form of an emulsion, another preferred embodiment of the manufacturing method according to the invention further comprises the additional steps of:

(e) drying the titrated and optionally processed vesicles obtained in step (c) or (d) in order to obtain lipid solid particles,

(f) mixing the lipid solid particles obtained in step (e) with an oily component,

(g) rising the temperature of the mixture obtained in step (f) above the melting temperature of the lipid solid particles obtained in step (e) but not until the temperature of degradation of the said lipids (i.e. usually not above about 80° C.), and

(h) emulsifying the mixture obtained in step (g) in the presence of water or a buffer.

Emulsions prepared according to the invention may be of any type, such as oil-in-water emulsions or water-in-oil emulsions.

Another embodiment of the manufacturing method of the invention further includes, after step (c) or (d), and optionally after admixing the lipid particles dispersion with a buffer (in the latter case, as explained above, a further pH control step is unnecessary in view of the advantageous pH characteristics of the dispersions of the invention), a step (i) of again processing the said lipid particles until a predetermined average size is obtained or until a predetermined size distribution is obtained. The processing method of this optional step, alike the processing methods of steps (b) and (d), is well known to those skilled in the art of liposomes and may be selected from any technology disclosed in Liposomes (cited supra), depending on the specific requirements of the further use of the liposomal dispersions, i.e. in particular depending from the desired mean size and mean sin distribution of the vesicles or particles in the dispersions. Such processing methods include micro-fluidization, vortex mixing, sonication and the like and make use of conventional manufacturing equipment available in the art. Depending upon the post-processing method selected for step (l), it is possible to achieve either multilamellar vesicles or small unilamellar vesicles or large unilamellar vesicles according to the classification of liposomes provided in the above section “Background of the Invention”.

In order to obtain the lipid particles dispersion in the form of amorphous solid particles (the latter having the advantages of a well controlled form and size), another preferred embodiment of the process according to the invention includes the following features:

(j) drying the titrated and optionally processed vesicles obtained in step (c) or (d) in order to obtain lipid solid particles,

(k) re-dispersing the solid particles obtained in step (j), optionally admixed with a biologically active molecule and/or with one or more lipids, in an organic solvent for compound A, the said solvent being sparingly miscible with water,

(l) processing the organic dispersion obtained in step (k), and

(m) stripping the organic solvent until amorphous solid particles are obtained.

The skilled person is readily able to select an organic solvent for compound A suitable for carrying out the above embodiment of the process according to the invention. Examples of suitable organic solvents for this purpose include for instance halogenated hydrocarbons such as chloroform and methylene chloride, esters such as ethyl acetate and mixtures thereof. The present invention thus also includes a composition of solid amorphous particles obtainable from the lipid particles dispersion by this embodiment of the manufacturing method of the invention.

According to yet another embodiment of the manufacturing method, a dried solid composition may be obtained from the dispersion of lipid particles of the invention by further including a step (n) of drying the titrated and optionally processed vesicles or liposomes obtained in step (c) or (d). When the drying step (h) is freeze-drying, the said dried solid composition is commonly named a lyophilisate. It has been checked that this post-titration drying step does not alter the advantageous characteristics of the product of the invention, i.e. physiological pH compatibility, even after re-dispersing the said dried solid composition or lyophilisate in water or a mineral buffer or an organic buffer.

In a third embodiment, the present invention further includes various uses of a liquid or solid dispersion of lipid particles according to the present invention, such as a solid composition (e.g. a composition of solid amorphous particles or a dried solid composition or lyophilisate) or an emulsion, including uses such as:

an ant-inflammatory agent or a component of an anti-inflammatory composition,

an anti-microbial agent or a component of an anti-microbial composition,

a cosmetic agent or a component of a cosmetic composition,

an emulsifier or a component of an emulsifying composition,

a detergent or a component of a detergent composition,

as a diagnostic reagent, and

as a vaccine adjuvant, especially for cytotoxic T lymphocyte induction, or a component of a vaccine composition; in the latter use, the dispersion of lipid particles of this invention may be admixed with an antigen and an immunopotentiatory amount of an immunogenicity inducing or enhancing compound; the vaccine composition may be administered orally, topically, epicutaneously, intramuscularly, intradermally, subcutaneously, intranasally, intravaginally, sublingually or via inhalation; for further details relating to such use, reference is made to “Vaccine adjuvants” (2000) ed. Derek T. O'Hagan, the content of which is incorporated herein by reference.

In all of the aforesaid uses, the lipid particle dispersion of this invention takes advantage of its pH compatibility characteristics. Additionally, the invention includes the use of a liquid or solid dispersion of lipid particles such as defined herein-above for the manufacture of a medicament, e.g. as an ingredient of a pharmaceutical or veterinary composition.

In a fourth embodiment, the present invention includes a synthetic vector or delivery vehicle characterised as being a combination of a dispersion of lipid particles such as previously disclosed and a biologically active molecule. Such synthetic vectors or delivery vehicles are useful in being able to introduce a wide range of biologically active molecules into a wide range of eukaryotic cells, preferably cells performing endocytosis or phagocytosis. In a first aspect of this embodiment, the biologically active molecule may be a therapeutic agent (such as defined hereinafter) which the lipid particles are able to transport over the membrane for introducing the said agent into the cell. In a second aspect of this embodiment, the biologically active molecule may be a macromolecule selected for instance from the group consisting of genetic material, polypeptides, glycosylated polypeptides, proteins, glycosylated proteins, protamine salts and sugars. Genetic material and cell hypes concerned by this aspect of the invention are listed in the section “Definitions” hereinabove. Importantly the present invention provides for efficient introduction of genetic material into a wide range of cell types, preferably cells performing endocytosis or phagocytosis, for instance fibroblast cells. Within such vectors and delivery vehicles, the weight ratio of the lipid particles dispersion to the said biologically active molecule is preferably from about 11:15 to 15:1, more preferably from 1:2 to 5:1. As is well known in the art, practical considerations such as toxicity at high concentrations, potentially adverse interactions with the biological milieu, side effects, ability to reach tissues and the like will dictate the selection of an appropriate weight ratio in each case, depending on the specific macromolecule concerned.

In a fifth embodiment, the present invention includes a method for introducing a biologically active molecule into a eukaryotic cell, comprising bringing said molecule in contact with a synthetic vector or delivery vehicle such as previously defined, in the presence of a culture medium containing the said eukaryotic cell. The type of cells concerned and the kind of macromolecules, especially genetic material, concerned in this embodiment are as disclosed with respect to the synthetic vectors hereinabove. When the said macromolecule is DNA, the biological material delivery method of the invention is preferably performed in the presence of a membrane permeability enhancing agent such as calcium phosphate. In another aspect of this embodiment, the present invention provides eukaryotic cells treated by the said method, i.e. transformed or transfected by means of a synthetic vector such as disclosed in detail hereinabove.

In a sixth embodiment, the present invention further includes a pharmaceutical composition comprising an effective amount of a dispersion of lipid particles (such as previously defined), optionally in combination with a biologically active molecule, and optionally one or more pharmaceutically acceptable carriers. Such pharmaceutical compositions are useful for administration in a therapeutically effective amount to a mammal, for instance a human, in need of the biologically active macromolecule included in the said composition. The invention also provides a method of treatment of a mammal in need of a biologically active molecule, comprising administering to the said mammal a therapeutically effective amount of the above pharmaceutical composition. According to standard practice in the art, administration to the patient may be effected by any conventional means, i.e. for instance orally, intranasally, subcutaneously, intramuscularly, intradermally, intravenously, intraarterially, parenterally or by catheterization,

As used in the previous embodiments of the present inventions the term “biologically active molecule” includes both therapeutic agents and cosmetic agents for topical or subcutaneous administration. Within the said meaning, the therapeutic agent may be selected from the group consisting of anti-fungal agents, hormones, vitamins, peptides, enzymes, polypeptides, glycosylated polypeptides, proteins, glycosylated proteins, anti-allergic agents, anti-coagulation agents, anti-tubercular agents, antiviral agents, antibiotics, anti-bacterial agents, anti-inflammatory agents, anti-protozoan agents, local anesthetics, growth factors, cardiovascular agents, diuretics and radioactive compounds. In particular, the therapeutic agent may be selected from the group consisting of scopolamine, nicotine, methylnicotinate, mechlorisone dibutyrate, naloxone, caffeine, salicylic acid, and 4-cyanophenol.

Suitable anti-fungal agents include ketoconazole, nystatin, griseofulvin, flucytosine, miconazole and amphotericin B. Suitable hormones include growth hormone, melanocyte stimulating hormone, estradiol, cortisol, luteinizing hormone, follicle stimulating hormone, somatotropin, somatomedins, adreno-corticotropic hormone, parathormone, vasopressin, thyroxine and testosterone. Suitable vitamins include retinoids, retinol palmitate, ascorbic acid and α-tocopherol. Suitable peptides and enzymes include bombesin, cholecystokinin, insulin: gastrin, endorphins, enkephalins, prolactin, oxytocin, gonadotropin, corticotropin, β-lipotropin, γ-lipotropin, calcitonin, glucagon, thyrtropin, elastin, cyclosporin, manganese super oxide dismutase and alkaline phoephatase. Suitable anti-coagulation agents include heparin. Suitable anti-tubercular agents include paraminosalicylic acid, isoniazid, capreomycin sulfate cycloserine, ethambutol hydrochloride ethionamnide, pyrazinamide, rifampin, and streptomycin sulfate. Suitable antiviral agents include acyclovir, amantadine, azidothymidine, ribavirin and vidarabine monohydrate. Suitable antibiotics include dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin, ticarillin, rifampin and tetracycline. Suitable anti-inflammatory agents include diflunisal, ibuprofen, indomethacin, meclofenamate, mefenamic acid, naproxen, oxyphenbutazone, phenylbutazone, piroxicam, sulindac, tolmetin, aspirin and salicylates. Suitable anti-protozoan agents include chloroquine, hydroxychloroquine, metronidazole, quinine and meglumine antimonate. Suitable local anesthetics include bupivacaine, chloroprocaine, etidocaine, lidocaine, mepivacaine, procaine and tetracaine and salts (such as hydrochloride) thereof. Suitable growth factors include Epidermal Growth Factor, Fibroblast Growth Factor, Insulin-Like Growth Factors, Nerve Growth Factor, Platelet-Derived Growth Factor, Stem Cell Factor, Transforming Growth Factors of the α family or the β family. Suitable cardiovascular agents include clonidine, propranolol, lidocaine, nicardipine and nitroglycerin. Suitable diuretics include mannitol and urea. Suitable radioactive compounds may include for instance a radioactive element selected from the group consisting of strontium, iodine, rhenium and yttrium.

Cosmetics suitable as biologically active molecules in this invention may be selected for instance from the group consisting of Vitamin A, Vitamin C, Vitamin D, Vitamin E, Vitamin K, β-carotene, collagen, elastin, retinoic acid, aloe vera, ointment bases (such as lanolin, squalene and the like), hyaluronic acid, sunscreen agents and nucleosides. Suitable sunscreen agents include for instance isobutyl p-aminobenzoate, diallyl trioleate, monoglyceryl p-aminobenzoate, propyleneglycol p-aminobenzoate, benzyl salicylate, benzyl cinnamate and mixtures thereof. When the biologically active molecule is a cosmetic agent, the pharmaceutical composition of the invention may take the form of a cosmetic cream, ointment, lotion, skin softener, gel, blush, eye-liner, mascara, acne-medication, cold cream, cleansing cream, or oleaginous foam.

Pharmaceutically acceptable carriers suitable for use in the pharmaceutical compositions of this invention include:

bacterlostatic agents such as quaternary ammonium compounds (including alkyldimethylbenzylammonium chlorides, cetylpyridinium chloride, cetyltrimethylammonium bromide, β-phenoxyethyldimethyldodecylammonium bromide and the like), benzoic acid, benzyl alcohol, p-hydroxybenzoic acid butyl ester or methyl ester, chlorobutanol, chlorocresol, phenol, potassium or sodium benzoate, potassium sorbate and sorbic acid;

antioxidants such as ascorbic acid and ascorbyl palmitate;

moisture content control agents and humecants;

suspending and viscosity-increasing agents such as agar, alginic acid, aluminum monostearate, bentonite, carbomers, carboxymethylcellulose calcium or sodium, carrageenan, microcrystalline cellulose, dextrin, gelatin, guar gum, hydroxyetylcellulose, hydroxypropylcellulose, hydroxypropylmethylcellulose, magnesium aluminum silicate, methylcellulose, pectin, polyethylene oxide, polyvinyl alcohol, polyvinylpyrrolidone, propylene glycol alginate, silicon dioxide, zinc oxide, sodium alginate tragacanth and xanthan gum;

skin absorption enhancing agents such as pyrrolidones, fatty acids, sulfoxides, amines, terpenes, terpenoids, urea, glycols and alcohols;

bases such as glycerol, propylene glycol, isopropyl myristate and polyethylene glycol; and

oleaginous vehicles, coloring agents or foaming agents.

The various embodiments of the present invention exhibit numerous a advantages over lipid dispersions of the prior art including:

the method for preparing the lipid particle dispersions of the invention is easy and inexpensive to implement and achieves liquid dispersions which have and retain a pH compatible with physiological pH within a temperature range (between about 2° C. and 40° C.) useful for most medical applications and which can easily be transformed into solid compositions, either dried (e.g. lyophilisates) or amorphous, or into emulsions retaining the latter property when re-dispersed in any physiological medium;

the lipid particle dispersions of the invention able to efficiently transfect a wide range of types of cells, especially calls performing endocytosis or phagocytosis; and

the lipid particle dispersions of the invention are therapeutically useful by themselves, either in vivo or in vitro, or can be included as formulation agents into a wide range of pharmaceutical compositions, diagnostic kits or cosmetic preparations.

The present invention will now be further explained by reference to the following working examples and comparative examples, which should in no way be interpreted as limiting its scope.

### EXAMPLE 1 COMPARATIVE

### Preparation of Amino-Amidine Liposomes in Water

N-terbutyl-N′-tetradecyl-3-tetradecyl-aminopropionamidine (having a melting point of 34° C.) is dispersed in water (injection grade available from BAXTER, Cat. Nr. ADA 0304) and kept overnight at 4° C. It is then dispersed at room temperature using a TV45 Ultra-Turrax blender (available from Jehnke & Kunkel) until a concentration of 3 mg/ml is achieved. The resulting dispersion was then poured into a M110S miorofluidizer (available from Microfluidics international Corp., Newton, Massachussetts) and then processed at 45° C. for four cycles of two minutes each, the interaction chamber outlet being packed in ice. The resulting liposomes were cooled and then passed through a 0.2 μm filter (in order to eliminate large particles) and then packed in sterile vials and stored at 4° C. Stability at 4° C. was satisfactorily checked by means of turbidity measurements for over five weeks.

### EXAMPLE 2

### Preparation and Control of Amidinium Salt Liposomes in Water

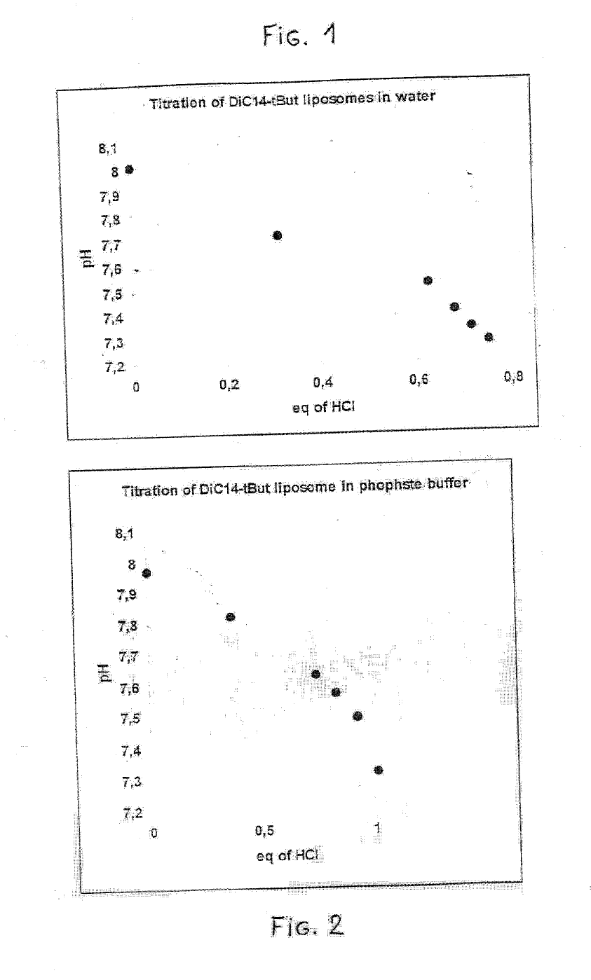

Immediately after the processing step disclosed in example 1, 1.1 mole equivalent of hydrochloric acid was added progressively to the microfluidized liposomes, pH of the solution being recorded at 25° C. as shown in FIG. 1.

After complete titration of the amidine function, the amidinium salt liposomes were processed again at 45° C. using the same M110S microfluidizer equipment as in example 1. At the end of this second processing ship, the pH of the dispersion was measured as 7.3 at 25° C.

The resulting titrated liposomes were cooled and then passed through a 0.2 μm filter (In order to eliminate large particles) and an average liposome size of 90 nm was measured. Filtered liposomes were then packed in sterile vials and stored at 4° C. Their stability at 4° C. was checked by turbidity measurements for over five weeks, i.e. titration by a strong acid had no adverse effect on the stability of the dispersion. Cytotoxicity was determined by means of a (3-(4,5-dimethylthiazol-2-yl)-2,5-diphenyl tetrazolium bromide assay, using a kit commercially available from Promega Benelux (Leyden, The Netherlands). COS-7 monkey fibroblast cell survival was expressed as the amount of dye reduction relative to that of the untreated control cells. The titrated liposomes are not toxic on COS-7 cells, as determined by the Cytotox 96 non-radioactive cytotoxy assay G 1780.

### EXAMPLE 3 COMPARATIVE

### Preparation of Amino-Amidine Liposomes in Sodium Phosphate Buffer

Preparation of liposomes was performed according to the procedure disclosed in example 1, except that the first step of N-terbutyl-N′-tetradecyl-3-tetradecylaminopropionamidine dispersion was effected in 20 mM sodium phosphate buffer (pH 7.3) instead of water.

### EXAMPLE 4

### Preparation and Control of Amidinium Salt Liposomes in Sodium Phosphate Buffer

Preparation of liposomes was performed according to the procedure disclosed in example 2 except that, alike in example 3, the initial processing step of amino-amidine dispersion was effected in 20 mM sodium phosphate buffer (pH 7.3) instead of water. The pH of the solution was recorded at 25° C. as a function of the amount of hydrochloric acid added, as shown in FIG. 2. Long-term (i.e. mare than five weeks) stability and cytotxicity of the titrated liposomes were successfully checked according to the same methodology as disclosed in example 2.

### EXAMPLE 5

### Transfection of Fibroblast Cells With Amino-Amidine Liposomes or Amidinium Salt Liposomes in the Presence of Protamine Sulfate

COS-7 monkey fibroblast cells were grown in a Dulbecco's Modified Eagle Medium (DMEM) culture medium supplemented with 10% heat-inactivated foetal bovine serum and antibiotic/antimytotic and maintained at 37° C. in a humidified 5% CO₂ incubator. The calls were then seeded, one day prior to transfection, in a 24-wells culture dish and allowed to reach at least 50%a confluency.

COS-7 monkey fibroblast cells were translated with DNA/protamine sulfate/amino-amidine complexes or DNA/protamine sulfate/amidinium salt complexes according to the following procedure. A plasmid DNA/protamine sulfate mixture (1:1 weight ratio) was mixed with either the amino-amidine liposomes of comparative examples 1 and 3 or the amidinium salt liposomes of examples 2 and 4, at a 1:2 DNA:lipids weight ratio in 20 mM sodium phosphate buffer at pH 7.3. After incubation at 23° C. for 15 minutes, 50 μl of the resulting DNA-lipid complex was mixed with 450 pi of DMFM and added to the cells for transfection. After two hours of incubation, the cell medium was changed with regular medium containing 10% heat inactivated foetal bovine serum (hereinafter referred as FBS). The cells were then incubated again for an additional 22 hours.

Functional transfection efficiency was then measured by means of a beta-galactosidase (hereinafter referred as β-GAL) activity assay as follows: cells were lysed by adding 250 μl of a lysis buffer (0.1 M potassium phosphate, 0.5% Triton® X-100, 0.1% deoxycholate, pH 7.0). The cell lysate (50 μl) was mixed with 50 μl o-nitrophenyl-β-D-galactopyranoside (1.54 mg/ml in 0.1 potassium phosphate buffer, pH 7.0) and incubated at 37° C. for 30 minutes. The reaction was terminated by adding 160 μl of 1 M Na₂CO₃ and absorbance was determined using a spectraphotometer at 405 nm. β-GAL activity was calculated using a β-GAL standard curve.

Table 1 indicates transfection efficiencies, expressed in IU (international units) per well, obtained for each of the liposomes prepared according to examples 1 to 4.

TABLE 1

| Example | 1 | 2 | 3 | 4 |
|---|---|---|---|---|
| IU/well | 0.0026 | 0.0589 | 0.0977 | 0.172 |

### EXAMPLE 6 COMPARATIVE

### Preparation of Mixed Amino-Amidine/Dimyristoylphosphatidyl Choline Liposomes in Sodium Phosphate Buffer

Preparation of liposomes was performed according to the procedure disclosed in example 3, except that N-terbutyl-N′-tetradecyl-3-tetradecylamino-propionamidine was replaced by a mixture comprising 50% by weight of the said amino-amidine and 50% by weight dimyristoylphosphatidyl choline.

### EXAMPLE 7

### Preparation of Mixed Amidinium Salt/Dimyristoylphosphatidyl Choline Liposomes in Sodium Phosphate Buffer

Preparation of liposomes was performed according to the procedure disclosed in example 4 except that, alike in example 6, N-terbutyl-N′-tetradecyl-3-tetradecylamino-propionamidine was replaced by a mixture comprising 50% by weight of the said amino-amidine and 50% by weight dimyristoylphosphatidyl choline. Long-term (i.e. more than five weeks) stability and cytotoxicity of the titrated mixed liposomes were successfully checked according to the same methodology as disclosed in example 2.

### EXAMPLE 8

### Transfection of Fibroblast Cells With Amino-Amidine Liposomes or Amidinium Salt Liposomes in the Absence of Protamine Sulfate

COS-7 monkey fibroblast cells were transfected according to the same experimental procedure as disclosed in example 5, except that: protamine sulfate was absent from the transfecting complexes.

Table 2 below indicates transfection efficiencies, measured as in example 5 and expressed in UI/well, obtained for each of the liposomes of examples 1-2 and 6-7.

TABLE 2

| Exemple | 1 | 2 | 6 | 7 |
|---|---|---|---|---|
| UI/well | 0.00267 | 0.058 | 0.0088 | 0.117 |

Results of examples 5 and 8 taken altogether clearly indicate that, whether in the presence or absence of protamine sulfate and whether in the presence or absence of a co-lipid in the liposomes, titration of the amidine function of the amino-amidine compound according to the procedure of the present invention makes it possible to multiply by a factor up to about 20 the transfection efficiency of DNA in fibroblast calls such as COS7 monkey cells.

1. A dispersion of lipid particles in a dispersing medium, the said lipid particles comprising an amino-amidine compound A having the general formula:
R₁HN—(CH₂)ₙ—C(═NR₂)—NR₃R₄
wherein each of R₁, R₂, R₃ and R₄ is independently selected from the group consisting of hydrogen, C₁₋₂₀ alkyl, C₃₋₁₀ cycloalkyl, aryl and heteroaryl radicals, and n is an integer form 1 to 6 inclusive, and the said lipid particles optionally further comprising one or more lipids B, wherein the amidine function of the said compound a is titrated substantially in water by means of an acid HX, wherein X is an anion, in a manner such that the pH of the said lipid dispersion is between about 6.5 and 7.8 within a temperature range from about 2° C. to 40° C.

2. A dispersion of lipid particles according to claim 1, wherein the said lipid particles are liposomes.

3. A dispersion of lipid particles according to claim 1, wherein the said lipid particles are emulsion droplets.

4. A dispersion of lipid particles according to claim 1, wherein the said lipid particles are solid particles.

5. A dispersion of lipid particles according to claim 1, wherein the dispersing medium comprises a mineral buffer which does not interfere with the pH of the said dispersion.

6. A dispersion of lipid particles according to claim 1, wherein the dispersing medium comprises an organic buffer being added after titration of the amidine function.

7. A method of making a dispersion of lipid particles, comprising the steps of:
(a) dispersing an amino-amidine compound A having the general formula
R₁HN—(CH₂)ₙ—C(═NR₂)—NR₃R₄
wherein each of R₁, R₂, R₃ and R₄ is independently selected from the group consisting of hydrogen, C₁₋₂₀ alkyl, C₃₋₁₀ cycloalkyl, aryl and heteroaryl radicals, and n is an integer from 1 to 6 inclusive, optionally admixed with one or more lipids B, in a liquid medium comprising an aqueous medium and optionally an organic solvent for compound A and for the lipid(s) B, and optionally an oily component,
(b) processing the dispersion obtained in step (a) until vesicles comprising compound A and optionally one or more lipid(s) B are obtained, and
(c) titrating the vesicles obtained in step (b) With an acid HX, wherein X is an anion, and
(d) optionally processing the titrated vesicles obtained in step (c) until pH stabilisation
the said method being characterized in that
the aqueous medium of step (a) substantially consists of water, and
the acid HX is used in step (c) in an amount such as to substantially form an amidinium salt (A, HX) and such that the pH of the said lipid dispersion after titration is between about 7.0 and 7.6 within a temperature range from about 2° C. to 40° C.

8. A method according to claim 7, further comprising the steps of,
(e) drying the titrated and optionally processed vesicles obtained in step (c) or (d) in order to obtain lipid solid particles,
(f) mixing the lipid solid particles obtained in step (e) with an oily component,
(g) rising the temperature of the mixture obtained in step (j) above the melting temperature of the lipid solid particles obtained in step (e) but not until the temperature of degradation of the said lipids (i.e. usually not above about 80° C.), and
(h) emulsifying the mixture obtained in step (g) in the presence of water or a buffer.

9. A method according to claim 7, further including after step (c) or (d) and optionally after admixing the lipid particles dispersion with a buffer, a step (i) of again processing the said lipid particles until a predetermined average size or a predetermined size distribution is obtained.

10. A method according to claim 7, further comprising the steps of:
(j) drying the titrated and optionally processed vesicles obtained in stop (c) or (d) in order to obtain lipid solid particles,
(k) re-dispersing the solid particles obtained in step (j), optionally admixed with a biologically active molecule and/or with one or more lipids, in an organic solvent for compound A, the said solvent being sparingly miscible with water,
(l) processing the organic dispersion obtained in step (k), and
(m) shipping the organic solvent until amorphous solid particles are obtained.

11. A dispersion of lipid particles according to claim 1, wherein the said amino-amidine is selected from the group consisting of N-terbutyl-N′-tetradecyl-3-tetradecylaminopropionamidine, N-terbutyl-N′-dodecyl-3-dodecylaminopropionamidine, N-terbutyl-N′-hexadecyl-3-hexadecylaminpropionamidine and N-terbutyl-N′-octadecyl-3-octadecylaminopropionamidine.

12. A dispersion of lipid particles according to claim 1 for use as an anti-inflammatory agent or an anti-microbial agent or a cosmetic agent or an emulsifier or a detergent or a vaccine adjuvant or a diagnostic reagent or a medicament.

13. A dispersion of lipid particles according to claim 1, wherein the said dispersion is combined with a biologically active molecule and optionally a pharmaceutically acceptable carrier.

14. A dispersion of lipid particles according to claim 1, wherein the said dispersion is combined with a biologically active molecule and optionally a pharmaceutically acceptable carriers, wherein the weight ratio of the dispersion of lipid particles to the biologically active molecule is from about 1:15 to 15:1.

15. A dispersion of lipid particles according to claim 1, wherein the said dispersion is combined With a biologically active molecule, wherein the said biologically active molecule is a macromolecule selected from the group consisting of nucleic acids, DNA, RNA, mRNA, rRNA, tRNA, uRNA, ribozymes, antisense oligonucleotides, peptide nucleic acid (PNA), plasmid DNA, polypeptides, glycosylated polypeptides, proteins, glycosylated proteins, protamine salts and sugars.

16. A dispersion of lipid particles according to claim 1, wherein the said dispersion is combined with a biologically active molecule, wherein the said biologically active molecule is a therapeutic agent or a cosmetic for topical or subcutaneous application.

17. A method for introducing a biologically active molecule into a eukaryotic cell, comprising bringing said biologically active molecule, in the presence of a culture medium containing the said eukaryotic cell, in contact with a dispersion of lipid particles according to claim 1.

18. A eukaryotic cell transformed by means of a combination of a biologically active macromolecule and a dispersion of lipid particles according to claim 1.

19. A method of treatment of a mammal in need of treatment, comprising administering to the said mammal a therapeutically effective amount of a pharmaceutical composition comprising an effective amount of a dispersion of lipid particles according to claim 1, optionally in combination with a biologically active molecule, and optionally one or more pharmaceutically acceptable carriers.
